(19) United States
(12) Patent Application Publication
(10) Pub. No.: US 2021/0145846 A1
(43) Pub. Date: May 20, 2021

(54) HIGH-CONCENTRATION FULVESTRANT COMPOSITIONS

(71) Applicant: Kashiv BioSciences, LLC, Bridgewater, NJ (US)

(72) Inventors: Navnit H. Shah, Clifton, NJ (US); Wantanee Phuapradit, Montville, NJ (US); Aruna Railkar, Wayne, NJ (US); Paras P. Jariwala, Somerset, NJ (US)

(21) Appl. No.: 16/322,733

(22) PCT Filed: May 22, 2018
(86) PCT No.: PCT/US2018/033871

§ 371 Date: Feb. 1, 2019

Related U.S. Application Data
(60) Provisional application No. 62/603,285, filed on May 23, 2017.
(60) Provisional application No. 62/646,618, filed on Mar. 22, 2018.

(51) Int. Cl. A61K 31/565; A61K 47/10; A61K 47/44; A61K 47/26; A61K 47/34; A61K 9/00

(52) U.S. Cl. CPC A61K 31/565; A61K 47/10; A61K 9/0019; A61K 47/26; A61K 47/34; A61K 47/44

(57) ABSTRACT

The present disclosure is directed to a pharmaceutical composition suitable for intramuscular (IM) administration, comprising fulvestrant, a pharmaceutically acceptable alcohol(s), a cosolvent(s), a lipophilic vehicle comprising a release-controlling oil, and, optionally, at least one surfactant and/or an antioxidant. The composition comprises fulvestrant at a concentration of at least 50 mg/ml, e.g., 100 mg/ml, wherein the composition exhibits excellent storage stability at long-term and accelerated storage conditions. The composition of the disclosure can deliver a 250-500 mg dose of fulvestrant as a single IM injection from a prefilled syringe, e.g., 500 mg/5 ml or 250 mg/2.5 ml in a prefilled syringe.

### CROSS-REFERENCE TO RELATED APPLICATIONS

This application claims priority to U.S. Provisional Patent Application Nos. 62/603,285, filed May 23, 2017, and 62/646,618, filed Mar. 22, 2018, the disclosures of which are hereby incorporated by reference herein in their entireties.

### TECHNICAL FIELD

The present disclosure relates to sustained release pharmaceutical compositions comprising fulvestrant at a concentration of at least 50 mg/mi, wherein the composition exhibits excellent storage stability under long-term and accelerated storage conditions, reduces muscular pain due to one intramuscular (IM) injection compared to two IM injections of currently marketed fulvestrant product. The composition of the disclosure can deliver, for example, a 250-500 mg dose of fulvestrant as a single intramuscular (IM) injection from a prefilled syringe (e.g., 250 mg/2.5 ml; 500 mg/5 ml prefilled syringes). The disclosure provides a fulvestrant composition comprising, in certain embodiments, fulvestrant; a solvent comprising a pharmaceutically acceptable alcohol(s); a cosolvent comprising propylene glycol; a release-controlling lipophilic vehicle comprising castor oil; and one or more surfactants comprising a lipophilic surfactant and/or a hydrophilic surfactant.

### BACKGROUND

Fulvestrant is a competitive estrogen receptor (ER) antagonist with an affinity comparable to that of estradiol, but without any agonist (estrogen-like) activity. Binding of fulvestrant to estrogen receptors has been shown to initiate and promote degradation of estrogen receptors, which ultimately blocks the cellular signal for estrogen synthesis.

FASLODEX® (AstraZeneca, NDA #21-344), a commercially available product of fulvestrant, is indicated for the treatment of hormone receptor (HR)-positive metastatic breast cancer in postmenopausal women with disease progression following antiestrogen therapy as monotherapy; and HR-positive, human epidermal growth factor receptor 2 (HER2)-negative, advanced or metastatic breast cancer, in combination with palbociclib, in women with disease progression after a first endocrine therapy.

A standard dose of 500 mg FASLODEX® IM is currently supplied as two 250 mg/5 ml prefilled syringes. The current dosing regimen of fulvestrant includes IM administration of a dose of 500 mg (as two 5 ml injections) of fulvestrant at days 1, 15, 29, and once monthly thereafter. A dose of 250 mg (as one 5 ml injection) of fulvestrant is recommended in patients with moderate hepatic impairment in the same fashion and at the same frequency.

Fulvestrant was initially approved as a 250 mg injection administered IM every month for treatment of HR-positive metastatic breast cancer in women with disease progression following antiestrogen therapy. However, subsequent evidence suggested that a fulvestrant dose of greater than 250 mg could provide enhanced pharmacodynamic activity in a sustained release pathway. Fulvestrant 500 mg was compared to 250 mg in a Phase III study (CONFIRM Phase III double-blind, parallel-group, multicenter clinical trial) using two injections of FASLODEX® for a 500 mg dose versus one injection for a 250 mg dose plus a placebo injection to appropriately blind the study. The results from this study showed that fulvestrant 500 mg provided a statistically significant increase in progression-free survival (PFS) with similar adverse events profile, without any associated increase in toxicity, corresponding to a clinically meaningful improvement, in benefit versus risk analysis, when compared with fulvestrant 250 mg. This benefit was further confirmed in a follow-up analysis in terms of overall survival. Median overall survival was 26.4 months for fulvestrant 500 mg compared with 22.3 months for fulvestrant 250 mg, indicating a clinically relevant difference in overall survival between the treatment groups (see, e.g., FASLODEX® label). Based on these results, in 2010, the U.S. Food and Drug Administration (FDA) changed the approved dose of fulvestrant from 250 mg IM to the current 500 mg IM, on days 1, 15, 29, and monthly thereafter.

Despite an increase in the approved dose, the IM formulation of fulvestrant, due to its limited solubility, is available only as a 250 mg/5 ml prefilled syringe composition. Therefore, for a 500 mg dose, a patient has to receive two 5 ml IM injections, one in each buttock, on days 1, 15 and 29 (a total of six injections in the first month to achieve a steady state) followed by additional 500 mg monthly doses (two injections monthly).

Injection site reaction is a frequently observed adverse event (AE) associated with fulvestrant injections, and such AEs have been shown to increase in number with an increase in the number of injections (Table 1). An interstudy comparison was performed to evaluate the effect of the number of injections on injection site reactions at the same total dose of fulvestrant. As shown in Table 1, the incidence of injection site reactions (AEs) increased from 7.3% to 27.0% corresponding to the increase in the number of injections from one to two; nevertheless, FASLODEX® is marketed as a two-injection dosing regimen. Similarly, it has been shown that the injection-site reactions lasted longer with the two 2.5 ml injection regimen compared to the one 5 ml injection (NDA #21-344) (FASLODEX® Reviews and Label, NDA #21-344, Medical/Statistical Review (s), page 93. CDER, 2002).

TABLE 1
Injection Site Reactions with FASLODEX ® at
250 mg dose administered either as one injection (250
mg/5 ml) or two injections (2 × 125 mg/2.5 ml) (NDA# 21-344)

| | | Patients | | | Courses | | |
|---|---|---|---|---|---|---|---|
| Trial # | Administration | Total | Events* | % | Total | Events* | % |
| 0020 | 5 ml × 1 dose | 219 | 16 | 7.3 | 1898 | 20 | 1.1 |
| 0021 | 2.5 ml × 2 doses | 204 | 55 | 27 | 1879 | 86 | 4.6 |

*Adverse events (AEs) = Local reactions at the injection site, consisting of pain, injection-site reaction, inflammation, and hemorrhage.

There is a need to develop a high concentration formulation of fulvestrant providing reduced injection site reactions and improved patient compliance. There is a need to develop a high-concentration formulation of fulvestrant with a goal of achieving a therapeutically equivalent exposure to FASLODEX® at a dose level of 500 mg/5 ml. Such high-concentration formulations may contain at least 100 mg/ml of fulvestrant and can improve patient experience and compliance by reducing the number of injections, and the total volume injected (e.g., 10 ml vs. 5 ml). Such high-concentration formulation can reduce injection site reactions and improve patient compliance, at a total dose of fulvestrant comparable to FASLODEX® by avoiding an extra injection each time, based on the presently approved schedule/dosing regimen.

### SUMMARY

In certain embodiments, the present disclosure provides a sustained release fulvestrant composition, suitable for IM administration, comprising fulvestrant; a solvent comprising a pharmaceutically acceptable alcohol(s); a cosolvent comprising propylene glycol, polyethylene glycol (one or more PEGs), and/or a nonaqueous ester; a lipophilic vehicle comprising castor oil; a hydrophilic and/or a lipophilic surfactant and, optionally, an antioxidant. The concentration of fulvestrant included in the composition is preferably from about 50 mg/ml to about 250 mg/ml.

In certain embodiments, the composition comprises fulvestrant at a concentration of at least about 50 mg/ml, e.g., 100 mg/ml, wherein the composition exhibits excellent storage stability, under extended storage conditions, e.g., at about 2-8° C., for a period of at least twelve months and can deliver a 500 mg dose of fulvestrant as a single IM injection of 5 ml or less.

In certain embodiments, the sustained release, high concentration fulvestrant composition of the disclosure is resistant to precipitation of fulvestrant and/or separation of castor oil and solvents/cosolvents as separate layers from the solution, upon storage for a period of at least about twelve months.

In certain embodiments, the composition, upon storage under standard storage conditions for at least about twelve months, is resistant to chemical degradation of fulvestrant.

In certain embodiments, the composition is resistant to precipitation of fulvestrant at the site of injection.

In certain embodiments, the sustained release fulvestrant composition of the disclosure provides resistance to degradation of fulvestrant, i.e., resistance to chemical degradation, upon storage at extended storage condition, e.g., at about 2-8° C., for a period of at least about twelve months, e.g., eighteen months, and can deliver a 500 mg dose of fulvestrant as a single IM injection of 5 ml or less.

In certain embodiments, the present disclosure provides a sustained release injectable composition comprising fulvestrant, wherein the composition provides an equivalent exposure to FASLODEX® at a dose level of 500 mg/5 ml, i.e., the composition in a concentration of a single dose comprising fulvestrant at 500 mg/5 ml concentration is bioequivalent to a combination of two doses of FASLODEX® comprising fulvestrant at 250 mg/5 ml concentration.

In certain embodiments, the present disclosure provides a sustained release injectable composition comprising fulvestrant; a pharmaceutically acceptable alcohol(s); a cosolvent comprising propylene glycol, a polyethylene glycol, benzyl benzoate, or a mixture thereof; a lipophilic surfactant, and/or a hydrophilic surfactant, an antioxidant, and a lipophilic vehicle comprising castor oil; wherein said pharmaceutical composition is bioequivalent to FASLODEX®.

In certain embodiments, the present disclosure provides a sustained release fulvestrant composition, wherein the composition exhibits excellent storage stability, e.g., the total impurities are below 2%, and 6-keto fulvestrant levels are comparable to FASLODEX®, when stored under similar conditions and time of storage, and the composition can deliver a 500 mg/5 ml dose of fulvestrant as a single IM injection.

In certain embodiments, the present disclosure provides a sustained release fulvestrant composition, wherein the composition can deliver 250 mg/2.5 ml dose of fulvestrant as a single IM injection.

In certain embodiments, the sustained release fulvestrant composition can deliver, for example, 1000 mg/10 ml dose of fulvestrant as two IM injections. In certain embodiments, the sustained release fulvestrant composition can deliver high doses of fulvestrant as two or more IM injections, wherein each IM injection contains 500 mg/5 ml dose of fulvestrant. In certain embodiments, the sustained release fulvestrant composition can deliver at least 500 mg/5 ml, e.g., 1000 mg/5 ml dose of fulvestrant in one IM injection.

In certain embodiments, the present disclosure provides a pharmaceutical composition comprising fulvestrant in an amount of at least 100 mg/mi; a pharmaceutically acceptable alcohol comprising a mixture of benzyl alcohol and ethanol; a cosolvent comprising propylene glycol; sorbitan monolaurate; poloxamer 188; dl-α-tocopherol; and a sufficient amount of lipophilic vehicle comprising castor oil, wherein the composition is free, or substantially free, of benzyl benzoate. In certain embodiments, the composition delivers a 500 mg/5 ml dose of fulvestrant as a single IM injection, and provides a therapeutically significant blood plasma fulvestrant concentration of at least about 2.5 ng/ml for at least about two weeks after injection.

In certain embodiments, the present disclosure provides a pharmaceutical composition suitable for IM administration comprising fulvestrant, a pharmaceutically acceptable alcohol(s), propylene glycol, a surfactant(s), and a lipophilic vehicle comprising castor oil. In certain embodiments, the composition comprises about 5% to about 20% w/v fulvestrant. In certain embodiments, the composition comprises fulvestrant at a concentration of at least 100 mg/ml in a single injection. In certain embodiments, the composition provides a fulvestrant concentration that is therapeutically equivalent to FASLODEX®, which is administered at a same total dose delivered in two injections. In certain embodiments, the pharmaceutically acceptable alcohol(s) comprises ethanol, benzyl alcohol, or a mixture thereof. In certain embodiments, the composition comprises at least 10% w/v of benzyl alcohol and at least 10% w/v of ethanol; or at least 10% w/v of benzyl alcohol and at least 15% w/v of ethanol. In certain embodiments, the surfactant is selected from the group consisting of a lipophilic surfactant, a hydrophilic surfactant, and mixtures thereof. In certain embodiments, the lipophilic surfactant is selected from the group consisting of sorbitan trioleate, sorbitan tristearate, sorbitan monooleate, sorbitan monostearate, sorbitan monopalmitate, and sorbitan monolaurate. In certain embodiments, the hydrophilic surfactant is selected from the group consisting of polyoxyethylene (20) sorbitan monolaurate, polyoxyethylene (20) sorbitan monopalmitate, polyoxyethylene (20) sorbitan monostearate, polyoxyethylene (20) sorbitan tristearate, polyoxyethylene (20) sorbitan monooleate, and a triblock copolymer of polyoxypropylene and polyoxyethylene (poloxamer 188). In certain embodiments, the composition further comprises about 0% to about 15% w/v of benzyl benzoate. In certain embodiments, the composition comprises at least 2% w/v of propylene glycol. In certain embodiments, the composition further comprises at least one antioxidant; in certain embodiments, the antioxidant is dl-α-tocopherol. In certain embodiments, the composition is physically and chemically stable, with no appreciable precipitation or degradation of fulvestrant, at about 5±3° C., for a period of at least six months; or for a period of at least 12 months. In certain embodiments, the composition is free, or substantially free, of benzyl benzoate. In certain embodiments, the composition is free, or substantially free, of antioxidant.

In certain embodiments, the present disclosure provides a pharmaceutical composition suitable for IM administration comprising: fulvestrant, a pharmaceutically acceptable alcohol(s), at least one cosolvent, a lipophilic surfactant, a hydrophilic surfactant, and a lipophilic vehicle comprising castor oil, wherein the composition is free, or substantially free, of benzyl benzoate and antioxidant. In certain embodiments, the pharmaceutically acceptable alcohol(s) comprises ethanol and benzyl alcohol. In certain embodiments, the cosolvent comprises propylene glycol, polyethylene glycol, or a mixture thereof. In certain embodiments, the hydrophilic surfactant is a triblock copolymer of polyoxypropylene and polyoxyethylene, and the lipophilic surfactant is sorbitan monolaurate.

In certain embodiments, the present disclosure provides a pharmaceutical composition suitable for IM administration comprising: fulvestrant, a pharmaceutically acceptable alcohol(s), at least one cosolvent, a surfactant, and a lipophilic vehicle comprising castor oil; wherein the composition is free, or substantially free, of benzyl benzoate and polysorbate 80. In certain embodiments, the pharmaceutically acceptable alcohol(s) comprises ethanol and benzyl alcohol. In certain embodiments, the cosolvent comprises propylene glycol, polyethylene glycol, or a mixture thereof. In certain embodiments, the cosolvent comprises propylene glycol. In certain embodiments, the surfactant comprises a hydrophilic surfactant comprising a triblock copolymer of polyoxypropylene and polyoxyethylene, and a lipophilic surfactant comprising sorbitan monolaurate.

In certain embodiments, the present disclosure provides a pharmaceutical composition suitable for IM administration comprising: fulvestrant, ethanol, benzyl alcohol, propylene glycol, a lipophilic surfactant, a hydrophilic surfactant, and a lipophilic vehicle comprising castor oil; wherein the composition is free, or substantially free, of benzyl benzoate and polysorbate 80.

In certain embodiments, the present disclosure provides a kit comprising at least one dosage form of an injectable pharmaceutical composition suitable for IM administration as provided by the present disclosure, and instructions for therapeutic administration of the at least one dosage form to a subject in need thereof. In certain embodiments, the pharmaceutical composition comprises fulvestrant, a pharmaceutically acceptable alcohol, propylene glycol, a surfactant, and a lipophilic vehicle comprising castor oil. In certain embodiments, the pharmaceutical composition comprises at least about 2% w/v of propylene glycol. In certain embodiments, the surfactant comprises a hydrophilic surfactant and a lipophilic surfactant. In certain embodiments, the lipophilic surfactant is sorbitan monolaurate, and the hydrophilic surfactant is a triblock copolymer of polyoxypropylene and polyoxyethylene. In certain embodiments, the pharmaceutical composition is packaged in a container, wherein the container comprises prefilled sterilized clear glass syringes, and wherein each syringe has a luer-lock (e.g., OVS®) tip cap, a sterilized plunger stopper, a plunger rod, and a sterilized needle. In certain embodiments, the composition comprises fulvestrant at a concentration of at least about 100 mg/ml in a single injection. In certain embodiments, the composition provides a therapeutically equivalent fulvestrant concentration to FASLODEX® at a same total dose delivered in two injections. In certain embodiments, the instructions for therapeutic administration comprise the steps of intramuscularly administering the contents of one prefilled syringe comprising the pharmaceutical composition to a subject in need thereof. In certain embodiments, the instructions provide a dosage regimen comprising single injections on each of Day 1, Day 15, Day 29, and once monthly thereafter.

In certain embodiments, the present disclosure provides a therapeutic method comprising intramuscularly administering to a subject in need thereof a pharmaceutical composition as provided in the present disclosure. In certain embodiments, the pharmaceutical composition comprises fulvestrant at a concentration of at least 100 mg/ml in a single injection, a pharmaceutically acceptable alcohol, propylene glycol, a surfactant, and a lipophilic vehicle comprising castor oil, wherein the composition provides a therapeutically equivalent fulvestrant concentration to FASLODEX® at a same total dose delivered in two injections, and wherein the administration of the composition achieves a reduction in adverse events, as compared to IM injections of FASLODEX®. In certain embodiments, the composition is free, or substantially free, of benzyl benzoate and antioxidant. In certain embodiments, the reduction in adverse events comprises reduction in pain, reduction in inflammation, and/or reduction in hemorrhage. In certain embodiments, the reduction in adverse events comprises reduction in duration of pain, reduction in duration of inflammation, and/or reduction in duration of hemorrhage.

In certain embodiments, the present disclosure provides a dosage regimen comprising intramuscularly administering to a subject in need thereof, a pharmaceutical composition as provided in the present disclosure. In certain embodiments, the pharmaceutical composition comprises fulvestrant, a pharmaceutically acceptable alcohol, propylene glycol, a surfactant, and a lipophilic vehicle comprising castor oil, wherein the dosing regimen comprises administering a single injection from a prefilled syringe on each of Day 1, Day 15, and Day 29, and once monthly thereafter, wherein each prefilled syringe comprises the pharmaceutical composition at a concentration of about 500 mg/5 ml, and wherein the composition provides a therapeutically equivalent fulvestrant concentration to FASLODEX® at the same total dose delivered in two injections. In certain embodiments, administration of the pharmaceutical composition results in a reduction in adverse events, wherein reductions in adverse events comprise reduction in pain, reduction in inflammation, and/or reduction in hemorrhage. In certain embodiments, the subject is a patient having a hormonal-dependent benign or malignant disease of the breast and/or reproductive tract. In certain embodiments, the subject is a postmenopausal woman with hormone receptor (HR)-positive metastatic breast cancer with disease progression following antiestrogen therapy. In certain embodiments, the subject is a postmenopausal woman with HR-positive, human epidermal growth factor receptor 2 (HER2)-negative, advanced or metastatic breast cancer, with disease progression after a first endocrine therapy. In certain embodiments, administration of the pharmaceutical composition results in improved therapeutic compliance by patients.

In certain embodiments, the present disclosure provides a method of stabilizing fulvestrant in solution comprising: 1) adding ethanolic solutions of sorbitan monolaurate, poloxamer 188, and an optional antioxidant to a vessel containing fulvestrant, benzyl alcohol and ethanol to obtain a clear solution, 2) adding a cosolvent comprising propylene glycol and/or polyethylene glycol to the clear solution from step #1, 3) adding a weighed amount of castor oil to the resulting solution from step #2 to obtain a final volume, 4) purging the vessel with nitrogen, and 5) filling the final volume into a 5 ml syringe. In certain embodiments, the final volume has a concentration of fulvestrant of at least 100 mg/ml. In certain embodiments, the composition is resistant to precipitation of fulvestrant and separation of castor oil, as a separate layer, from the solvent/cosolvent mixture for a period of at least about eighteen months.

In certain embodiments, the present disclosure provides a sterile prefilled, clear glass syringe comprising a composition for intramuscular administration, as provided in the present disclosure. In certain embodiments, the composition comprises fulvestrant in an amount of at least 100 mg/ml, a pharmaceutically acceptable alcohol(s), a cosolvent, a hydrophilic surfactant, a lipophilic surfactant, and a lipophilic vehicle comprising castor oil, wherein the composition is free, or substantially free, of benzyl benzoate.

In certain embodiments, the present disclosure provides a method for decreasing the frequency by which a muscle site is subject to intramuscular injection comprising administering an improved fulvestrant pharmaceutical composition, as provided in the present disclosure. In certain embodiments, the pharmaceutical composition comprises fulvestrant, a pharmaceutically acceptable alcohol(s), propylene glycol, a surfactant comprising a lipophilic surfactant and a hydrophilic surfactant, and a lipophilic vehicle comprising castor oil, wherein the concentration of fulvestrant in the composition is at least about 100 mg/ml.

In certain embodiments, the present disclosure provides a method for improving patient compliance in treating HR-positive metastatic breast cancer comprising administering a pharmaceutical composition, as provided in the present disclosure. In certain embodiments, the pharmaceutical composition comprises fulvestrant as a single IM injection, wherein the pharmaceutical composition comprises fulvestrant, a pharmaceutically acceptable alcohol, propylene glycol, a surfactant comprising a lipophilic surfactant and a hydrophilic surfactant, and a lipophilic vehicle comprising castor oil, and wherein the composition contains fulvestrant in an amount of about 100 mg/ml.

In certain embodiments, the present disclosure provides a pharmaceutical composition comprising fulvestrant in an amount of at least about 100 mg/ml; benzyl alcohol; ethanol; propylene glycol; sorbitan monolaurate; poloxamer 188; and a sufficient amount of castor oil vehicle, wherein the composition is free, or substantially free, of benzyl benzoate and antioxidant, and wherein the composition delivers a 500 mg/5 ml dose of fulvestrant as a single IM injection.

### DETAILED DESCRIPTION

### Definitions

The terminology used in the present disclosure is for the purpose of describing particular embodiments only and is not intended to be limiting. As used in the description of the embodiments of the disclosure and the appended claims, the singular forms “a,” “an,” and “the” are intended to include the plural forms as well, unless the context clearly indicates otherwise.

The term “and/or,” as used herein, refers to and encompasses any and all possible combinations of one or more of the associated listed items.

The term “about,” as used herein when referring to a measurable value such as an amount of a compound, dose, time, temperature, and the like, is meant to encompass variations of 20%, 10%, 5%, 1%, 0.5%, or even 0.1% of the specified amount. Unless otherwise defined, all terms, including technical and scientific terms used in the description, have the same meaning as commonly understood by one of ordinary skill in the art to which this disclosure belongs.

The term “therapeutically significant level,” “therapeutically significant blood plasma fulvestrant concentration,” and the like, as used herein, is meant to encompass blood plasma concentrations of fulvestrant at least 2.5 ng/ml, e.g., at least 3.0 ng/ml, 4.0 ng/ml, 5.0 ng/ml, 6.0 ng/ml, 7.0 ng/ml, 8.0 ng/ml, 9.0 ng/ml, 10.0 ng/ml, 11.0 ng/ml, or 12.0 ng/ml.

The term “therapeutically equivalent,” as used herein, is meant to encompass compositions which can be expected to have substantially the same clinical effect and/or safety profile when administered to patients under substantially similar conditions.

The term “sustained release” as used herein, are used to encompass at least three weeks (e.g., at least four weeks) of continuous/extended release of fulvestrant. In certain embodiments, sustained release is achieved for at least 28 days. In certain embodiments, sustained release of fulvestrant is for at least 2-5 weeks, e.g., at least 2.5-5, 2.5-4, 3-4, 3.5-4, or 4 weeks.

The term “lipophilic surfactant” as used herein with respect to a composition is meant to encompass surfactants with a hydrophilic-lipophilic balance (HLB) of between 1 and 10.

The term “hydrophilic surfactant” as used herein with respect to a composition is meant to encompass surfactants with an HLB of 10 and above.

The term “hydrophilic-lipophilic balance” (HLB) describes the balance of the size and strength of the hydrophilic and lipophilic groups.

The term “lipophilic vehicle” as used herein with respect to a composition is meant to encompass lipophilic viscous compounds (e.g., viscosity of at least 500 cp) providing a sustained release profile of the disclosed formulations.

The term “stability” as used herein with respect to a composition is meant to encompass any characteristics of a composition that may be affected by long term and accelerated storage conditions including, without limitation, total impurities, fulvestrant degradation products, specific optical rotation, optical purity, water content, appearance (including precipitation of fulvestrant or other excipients present in the formulation), and/or layer separation of hydrophilic and lipophilic components, separation of the lipophilic vehicle from the solvent/cosolvent (e.g., layer separation), viscosity, sterility, color, and clarity. Methods for determining the stability of a composition with respect to the above parameters include, without limitation, physical appearance of the composition, and high-performance liquid chromatography (HPLC) or thin layer chromatography (TLC).

The term “long-term storage conditions” as used herein includes storage in clear type 1 glass prefilled syringes with polystyrene plunger rods, fitted with tamper-evident closures, each containing 500 mg/5 ml of fulvestrant solution for IM injection; storage/transport is at 5±3° C.

The term “accelerated storage conditions” as used herein includes storage in clear type 1 glass prefilled syringes with polystyrene plunger rods, fitted with tamper-evident closures, each containing 5 ml of fulvestrant solution for IM injection; storage/transport is at 25±2° C. and at 60±5% relative humidity.

### Formulation

The disclosure provides a sustained release pharmaceutical formulation comprising fulvestrant at greater than about 50 mg/ml concentration (e.g., 100 mg/ml, 150 mg/ml concentration), wherein the composition exhibits excellent storage stability under long-term and accelerated storage conditions and can deliver, for example, a 500 mg dose of fulvestrant as a single IM injection. In certain embodiments, the disclosure provides fulvestrant compositions comprising fulvestrant at 100 mg/ml concentration; a pharmaceutically acceptable alcohol(s); a cosolvents) comprising a propylene glycol, polyethylene glycol, and/or a nonaqueous ester solvent; a release controlling lipophilic vehicle comprising castor oil; and, optionally, at least one antioxidant and/or a surfactant comprising a lipophilic surfactant and/or a hydrophilic surfactant.

Fulvestrant (7α-[9-[(4,4,5,5,5-pentafluoropentyl)sulfinyl]nonyl]-estra-1,3,5(10)-triene-3,17β-diol) has the following structural formula:

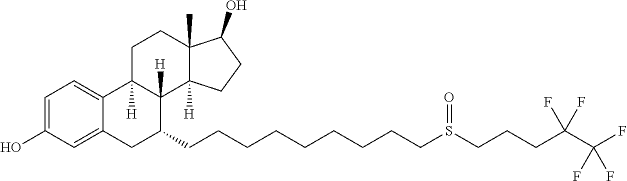

Fulvestrant contains six asymmetric carbon atoms and a stereogenic sulfoxide in the side chain. Fulvestrant used in the FASLODEX® injection product is a mixture of two diastereoisomers, which are referred to in the art as fulvestrant sulfoxide A and B. Fulvestrant sulfoxide A and B have the same absolute configuration at each of the asymmetric centers in the steroid system, but different absolute configurations at the sulfur atom. In certain embodiments of the disclosure, the fulvestrant can be fulvestrant sulfoxide A, fulvestrant sulfoxide B, or a mixture of fulvestrant sulfoxide A and fulvestrant sulfoxide B. In addition, fulvestrant can be in free form, or fulvestrant can be in salt or solvate form, such as a pharmaceutically acceptable fulvestrant salt or fulvestrant solvate. All salt and nonsalt forms of fulvestrant, and solvates of the foregoing, are embraced by the disclosure and the descriptions of fulvestrant provided herein. The fulvestrant can be crystalline or amorphous. If crystalline, fulvestrant can be any polymorphic form.

In certain embodiments, the amount of fulvestrant present in the composition is about 5% w/v (weight per volume of the composition) or more, e.g., about 6%, 7%, 8%, 9%, 10%, 11%, 12%, 13%, 14%, 15%, 16%, 17%, 18%, 19%, 20%, 21%, 22%, 23%, 24%, or 25% w/v (or intervening amounts/percentages). In certain embodiments, the amount of fulvestrant present in the composition is in a range bounded by any of the foregoing values. For example, the amount of fulvestrant present in the composition can be about 5-25%, 6-22%, 7-19%, 8-15%, or 9-11% w/v. In certain embodiments, the amount of fulvestrant present in the composition is at least about 10% w/v, e.g., 100 mg/ml (i.e., 500 mg/5 ml). In certain embodiments, the amount of fulvestrant present in the composition is at least about 150 mg/ml, 200 mg/ml, or 250 mg/ml, or intermediate concentrations thereof.

To develop a high-concentration formulation of fulvestrant, an extensive solubility assessment was performed based on the published literature as well as laboratory experiments using various solvents, cosolvents, and excipients, including surfactants and lipids. An initial assessment based on the literature data is shown in Table 2.

TABLE 2
Solubility of Fulvestrant in Various Solvents/Excipients

| | Solubility |
|---|---|
| | (mg/ml) |
| Solvent/Excipient | at 25° C. |
| Sesame oil | 0.58 |
| Castor oil | 20 |
| Migloyl 810 | 3.06 |
| Migloyl 812 | 2.72 |
| Ethyl oleate | 1.25 |
| Benzyl benzoate | 6.15 |
| Isopropyl myristate | 0.80 |
| SPAN ® 85 | 3.79 |
| Ethanol | >200 |
| Benzyl alcohol | >200 |
| Propylene glycol | 4.0 |
| Polyethylene glycol (PEG 400) | 22.5 |

In certain embodiments, benzyl alcohol and ethanol were selected as the solvents, and castor oil was selected as a lipophilic vehicle to provide sustained release profile of the formulation. In certain embodiments, at least one surfactant, e.g., a mixture of at least one hydrophilic surfactant and at least one lipophilic surfactant, was included to achieve and maintain high stability of the formulation, e.g., storage stability without precipitation of fulvestrant, or separation of lipophilic vehicle from the solvent(s)/cosolvent(s); and spreadability of the formulation. In certain embodiments, cosolvents, such as benzyl benzoate, PEG, propylene glycol, or mixtures thereof, were included to provide a viscosity and an overall consistency to the formulation for a desired release profile.

In certain embodiments, the amounts of solvents (e.g., ethanol and benzyl alcohol) and cosolvents (e.g., propylene glycol) were adjusted/fine-tuned to provide a therapeutically equivalent fulvestrant concentration in a single injection to that of FASLODEX® at the same total dose delivered in two injections. Examples 1-3 show several variations in the amounts of solvents (e.g., ethanol and benzyl alcohol) and cosolvents (e.g., propylene glycol, polyethylene glycol, and benzyl benzoate) in experimental formulations that were studied/examined to provide a therapeutically equivalent fulvestrant concentration/single injection to that of FASLODEX® at the same total dose delivered in two injections. In certain embodiments, the amounts of solvents and cosolvents were adjusted to provide an AUC (area under curve) of the fulvestrant composition comprising 10% w/v of fulvestrant similar to that of FASLODEX®.

In certain embodiments, a blend of at least one lipophilic surfactant and at least one hydrophilic surfactant was used rather than a hydrophilic surfactant alone or a lipophilic surfactant alone. In certain embodiments, the use of such blend of a hydrophilic surfactant and a lipophilic surfactant provided an improved solubilizer for the set of ingredients and conditions used in some fulvestrant compositions of the disclosure. In certain embodiments, polyvinyl pyrrolidone was used as a solubilizer/precipitation inhibitor. In certain embodiments, polyvinyl pyrrolidone was used with at least one surfactant.

The fulvestrant IM formulation is a viscous, oily solution. Once injected into the muscle tissue, it forms a depot and the formulation components begin to diffuse into the surrounding tissue and the circulatory system. The rate of diffusion for each component depends on the solubility, permeability, and viscosity of the formulation in the local environment of the depot. Notwithstanding the complexity of the in vivo situation, and without wishing to be held to a particular theory, it is expected that the viscosity of the formulation is a critical factor that affects the diffusion of the formulation components from the site of injection, including from the point of view of ease of injection, diffusion into the muscle tissue, and rate of absorption. Viscosities of the components in the prototype formulations are shown in Table 3. Based on the significant differences in the viscosity of cosolvents and the lipid vehicle, it is expected that their relative concentrations in the formulation can be critical not only for solubility but also for performance of the formulations, e.g., release profile of the formulation.

TABLE 3

| Excipient | Viscosity (cp) |
|---|---|
| Ethyl alcohol (Ethanol) | 1.04 |
| Benzyl alcohol | 5.40 |
| Benzyl benzoate | 8.90 |
| Propylene glycol | 42.00 |
| PEG 400 | 90.00 |
| Castor oil | 660.00 |
| SPAN ® 20 | 1000-2000 |

In certain embodiments, viscosities of the formulations were determined using a Brookfield viscometer. In certain embodiments, the viscosities were determined using a Rheosense viscometer. In certain embodiments, the viscosity of a fulvestrant formulation of the disclosure is at least about 40 cp, e.g., about 45 cp, 50 cp, 55 cp, 60 cp, 65 cp, 70 cp, 75 cp, 80 cp, 85 cp, 90 cp, 95 cp, 100 cp, 105 cp, 110 cp, 115 cp, 120 cp, 125 cp, or 130 cp. In certain embodiments, the viscosity of the fulvestrant formulation is between about 80 cp and about 150 cp, e.g., between about 85 cp and about 120 cp.

In certain embodiments, the disclosure provides a pharmaceutical formulation composition comprising fulvestrant at 100 mg/ml dose, pharmaceutically acceptable alcohols comprising ethanol and benzyl alcohol, a cosolvent(s) comprising benzyl benzoate and/or propylene glycol, a lipophilic surfactant, a hydrophilic surfactant, an antioxidant, and a lipophilic vehicle comprising castor oil, wherein the composition provides a sustained release of therapeutically significant levels of fulvestrant.

In certain embodiments, the pharmaceutically acceptable alcohol can be one alcohol or a mixture of two or more alcohols. In certain embodiments, the pharmaceutically acceptable alcohol is a mixture of two alcohols. In certain embodiments, pharmaceutically acceptable alcohol(s) includes, without limitation, ethanol, benzyl alcohol, or a mixture of both ethanol and benzyl alcohol.

In certain embodiments, the amount of pharmaceutically acceptable alcohol(s) present in the composition is about 5% w/v or more, e.g., about 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35% w/v, or intermediate percentages thereof.

In certain embodiments, the pharmaceutically acceptable alcohol(s) comprises ethanol and benzyl alcohol.

In certain embodiments, the amount of ethanol present in the composition is about 5% w/v or more, e.g., about 6%, 7%, 8%, 9%, 10%, 11%, 12%, 13%, 14%, 15%, 16%, 17%, 18%, 19%, 20%, 21%, 22%, 23%, 24%, 25%, 26%, or 27% w/v, or intermediate percentages thereof. In certain embodiments, the composition comprises about 10-25% w/v of ethanol

In certain embodiments, the amount of benzyl alcohol present in the composition is about 5% w/v or more, e.g., about 6%, 7%, 8%, 9%, 10%, 11%, 12%, 13%, 14%, 15%, 16%, 17%, 18%, 19%, 20%, 21%, 22%, 23%, 24%, or 25% w/v, or intermediate percentages thereof. In certain embodiments, the composition comprises about 5-20% w/v of benzyl alcohol. In certain embodiments, the composition comprises at least about 10% w/v of benzyl alcohol.

In certain embodiments, the cosolvent comprises a nonaqueous ester, dimethyl sulfoxide (DMSO), glycofurol, a PEG, propylene glycol, or mixtures thereof. In certain embodiments, the pharmaceutically acceptable nonaqueous ester may consist of one, or a mixture of two or more, pharmaceutically acceptable nonaqueous esters. In certain embodiments, a pharmaceutically acceptable nonaqueous ester for parenteral (e.g., IM) administration is selected from benzyl benzoate, ethyl oleate, isopropyl myristate, isopropyl palmitate, or a mixture thereof. In certain embodiments, no cosolvent is added to the formulation.

In certain embodiments, the cosolvent comprises a nonaqueous ester, e.g., benzyl benzoate. In certain embodiments, the amount of nonaqueous ester, e.g., benzyl benzoate, present in the composition is less than about 10% w/v, e.g., less than about 9%, 8%, 7%, 6%, 5.5%, 5%, 4.5%, 4%, 3.5%, 3%, 2.5%, 2%, 1.5%, 1%, or 0.5% w/v, or intermediate percentages thereof. In certain embodiments, the composition contains no benzyl benzoate, e.g., is free of benzyl benzoate.

In certain embodiments, the cosolvent comprises propylene glycol. In certain embodiments, the amount of propylene glycol present in the composition is less than about 10% w/v, e.g., less than about 9%, 8%, 7%, 6%, 5.5%, 5%, 4.5%, 4%, 3.5%, 3%, 2.5%, 2%, 1.5%, 1%, or 0.5%, or intermediate percentages thereof. In certain embodiments, the composition comprises about 1-10% w/v of propylene glycol. In certain embodiments, the composition comprises at least about 2% w/v of propylene glycol. In certain embodiments, the amount of propylene glycol present in the composition is 0% w/v.

In certain embodiments, the cosolvent comprises a mixture of benzyl benzoate and propylene glycol. In certain embodiments, the total amount of benzyl benzoate and propylene glycol present in the composition is less than about 15%, e.g., less than about 14%, 13%, 12%, 11%, 10%, 9%, 8%, 7%, 6%, 5.5%, 5%, 4.5%, 4%, 3.5%, 3%, 2.5%, 2%, 1.5%, 1%, or 0.5% w/v, or intermediate percentages thereof. In certain embodiments, the total amount of benzyl benzoate and/or propylene glycol present in the composition is 0% w/v.

In certain embodiments, the cosolvent comprises a mixture of propylene glycol and a PEG (e.g., PEG 400). In certain embodiments, the total amount of propylene glycol and a PEG present in the composition is less than about 15%, e.g., less than about 14%, 13%, 12%, 11%, 10%, 9%, 8%, 7%, 6%, 5.5%, 5%, 4.5%, 4%, 3.5%, 3%, 2.5%, 2%, 1.5%, 1%, or 0.5% w/v, or intermediate percentages thereof. In certain embodiments, the total amount of propylene glycol and a PEG present in the composition is 0% w/v.

In certain embodiments, the cosolvent comprises a mixture of benzyl benzoate and a PEG (e.g., PEG 400). In certain embodiments, the total amount of benzyl benzoate and a PEG present in the composition is less than about 15%, e.g., less than about 14%, 13%, 12%, 11%, 10%, 9%, 8%, 7%, 6%, 5.5%, 5%, 4.5%, 4%, 3.5%, 3%, 2.5%, 2%, 1.5%, 1%, or 0.5% w/v, or intermediate percentages thereof. In certain embodiments, the total amount of benzyl benzoate and a PEG present in the composition is 0% w/v.

In certain embodiments, it has been surprisingly found that the inclusion of a mixture of at least one hydrophilic surfactant and at least one lipophilic surfactant in a castor oil-based composition provides a surfactant blend compatible with fulvestrant and castor oil, and with suitable HLB balance to facilitate solubilization of fulvestrant at a concentration of 100 mg/ml or greater with about 5% w/v or less of cosolvents. In certain embodiments, the fulvestrant composition of the disclosure includes at least one surfactant and/or a solubility enhancer. In certain embodiments, the solubility enhancer is polyvinyl pyrrolidone. In certain embodiments, this combination of surfactants in the formulation can reduce or eliminate precipitation of fulvestrant and improve spreadability of the formulation upon injection. In certain embodiments, upon administration to a subject, the composition of the disclosure advantageously provides a pharmacokinetic profile similar to that provided by the FASLODEX® injection product.

In certain embodiments, the amount of a hydrophilic surfactant present in the composition is in the range of about 0.05% to about 0.35% w/v. In certain embodiments, the amount of a hydrophilic surfactant present in the composition is about 0.05% w/v or more, e.g., about 0.08%, 0.1%, 0.15%, 0.2%, 0.25%, 0.3%, or 0.35 w/v, or intermediate percentages thereof. In certain embodiments, the composition comprises about 0.1-0.3% w/v of hydrophilic surfactant. In certain embodiments, the composition comprises at least about 0.2% w/v of hydrophilic surfactant. In certain embodiments, the amount of poloxamer 188 present in the composition is about 0.05% w/v or more, e.g., about 0.08%, 0.1%, 0.15%, 0.2%, 0.25%, 0.3%, or 0.35 w/v, or intermediate percentages thereof.

In certain embodiments, the amount of a lipophilic surfactant present in the composition is in the range of about 0.05% to about 0.35% w/v. In certain embodiments, the amount of a lipophilic surfactant present in the composition is about 0.05% w/v or more, e.g., about 0.08%, 0.1%, 0.15%, 0.2%, 0.25%, 0.3%, or 0.35 w/v, or intermediate percentages thereof. In certain embodiments, the amount of sorbitan monolaurate present in the composition is about 0.05% w/v or more, e.g., about 0.08%, 0.1%, 0.15%, 0.2%, 0.25%, 0.3%, or 0.35 w/v, or intermediate percentages thereof.

In certain embodiments, the composition comprises at least one pharmaceutically acceptable antioxidant. In certain embodiments, the antioxidant is present in an amount necessary to suppress the formation of oxidative degradation products. Suitable antioxidants and general concentration ranges are described, for example, in “Remington: The Science and Practice of Pharmacy,” 21st edition, ed. P. Beringer, Lippincott Williams & Wilkins (2005) and in “Handbook of Pharmaceutical Excipients,” 7th edition, ed. R. Rowe, Pharmaceutical Press (2012).

In certain embodiments, the antioxidant is selected from the group consisting of a vitamin E compound, lipoic acid, dihydrolipoic acid, methionine, butylated hydroxytoluene (BHT), butylated hydroxyanisole, (BHA), and sodium formaldehyde sulfoxylate. In certain embodiments, the antioxidant is a vitamin E compound, including one or more of α, β, γ, or δ-tocopherol, and/or α, β, γ, or δ-tocotrienol. The tocopherol or tocotrienol can be a d-tocopherol or d-tocotrienol (2R configuration), an 1-tocopherol or 1-tocotrienol (2S configuration), or a mixture of a d,l-tocopherol and/or d,l-tocotrienol.

In certain embodiments, the antioxidant is dl-α-tocopherol. In certain embodiments, the amount of antioxidant present in the composition is less than about 0.5%, e.g., about 0.4%, 0.35%, 0.3%, 0.25%, 0.2%, 0.15%, 0.1%, 0.09%, 0.08%, 0.075%, 0.07%, 0.065%, 0.06%, 0.055%, 0.05%, 0.045%, 0.04%, 0.035%, 0.03%, 0.025%, 0.02%, 0.015%, 0.01%, 0.009%, 0.008%, 0.007%, 0.006%, 0.005%, 0.004%, 0.003%, 0.002%, 0.001%, or 0.0% w/v, or intermediate percentages thereof.

In certain embodiments, the composition is free of antioxidant. It has surprisingly and unexpectedly been observed that the presence of an antioxidant in the composition did not enhance the storage stability of the composition. It was also surprisingly observed that dl-α-tocopherol, used for its antioxidant properties, was unstable and susceptible to degradation under standard and accelerated storage conditions.

In certain embodiments, the vehicle of the sustained release fulvestrant formulation is a lipophilic vehicle. In certain embodiments, the lipophilic vehicle is a ricinoleate vehicle. In certain embodiments, the ricinoleate vehicle is castor oil. In certain embodiments, the amount of castor oil present in the composition is about 30% w/v or more, e.g., about 40%, 50%, 60%, 65%, or 70% w/v or more, or intermediate percentages thereof.

In certain embodiments, the composition of the disclosure may further include, but is not limited to, diluents, stabilizers, solubilizers, and preservatives.

In certain embodiments, the composition of the disclosure comprising fulvestrant; a pharmaceutically acceptable alcohol(s), for example, ethanol and/or benzyl alcohol; one or more cosolvents comprising a nonaqueous ester, a PEG, and/or propylene glycol; a lipophilic vehicle comprising castor oil; and, optionally, an antioxidant and/or a surfactant comprising a hydrophilic surfactant and/or a lipophilic surfactant, can be prepared by any suitable method.

In certain embodiments, the cosolvent comprises a nonaqueous ester. In certain embodiments, the cosolvent comprises a PEG. In certain embodiments, the cosolvent comprises propylene glycol. In certain embodiments, the cosolvent comprises a nonaqueous ester and a PEG. In certain embodiments, the cosolvent comprises a nonaqueous ester and propylene glycol. In certain embodiments, the cosolvent comprises a PEG and propylene glycol. In certain embodiments, the cosolvent comprises a nonaqueous ester, a PEG, and propylene glycol. In certain embodiments, the PEG is PEG 400. In certain embodiments, the nonaqueous ester is benzyl benzoate. In certain embodiments, the composition comprises no cosolvent.

In certain embodiments, the composition comprises a hydrophilic surfactant. In certain embodiments, the composition comprises a lipophilic surfactant. In certain embodiments, the composition comprises a mixture of hydrophilic and lipophilic surfactants. In certain embodiments, the composition comprises no surfactant, e.g., is free of surfactant.

In certain embodiments, the lipophilic surfactants comprise sorbitan trioleate (SPAN® 85), sorbitan tristearate (SPAN® 65), sorbitan monooleate (SPAN® 80), sorbitan monostearate (SPAN® 60), sorbitan monopalmitate (SPAN® 40), and sorbitan monolaurate (SPAN® 20). In certain embodiments, the lipophilic surfactant is sorbitan monolaurate (SPAN® 20).

In certain embodiments, the hydrophilic surfactants comprise polyoxyethylene (20) sorbitan monolaurate (TWEEN® 20), polyoxyethylene (20) sorbitan monopalmitate (TWEEN® 40), polyoxyethylene (20) sorbitan monostearate (TWEEN® 60), polyoxyethylene (20) sorbitan tristearate (TWEEN® 65), polyoxyethylene (20) sorbitan monooleate (TWEEN® 80), and a triblock copolymer of polyoxypropylene and polyoxyethylene (poloxamer 188; e.g., SYNPERONIC® F68). In certain embodiments, the hydrophilic surfactant is a triblock copolymer of polyoxypropylene and polyoxyethylene (poloxamer 188).

In certain embodiments, the composition is formed by combining the components in a suitable vessel. In certain embodiments, separate ethanolic solutions of SPAN® 20, dl-α-tocopherol, poloxamer 188, and fulvestrant are added to a vessel containing benzyl alcohol to obtain a clear solution, followed by addition of cosolvent(s) comprising benzyl benzoate and/or propylene glycol. In certain embodiments, ethanolic solutions of sorbitan monolaurate and poloxamer 188 are added to a solution of fulvestrant dissolved in benzyl alcohol and/or ethanol. To the resulting solution, a weighed amount of castor oil is added to obtain a final volume. In certain embodiments, the vessel is pressurized with nitrogen upon addition of castor oil.

In certain embodiments, the composition is filtered through one or more filters prior to filling the composition into one or more suitable containers, e.g., a vial, an ampoule, a cartridge, or a syringe. The filtration step and the filling step are performed under aseptic conditions in order to provide sterile conditions.

In certain embodiments, the disclosure provides a prefilled syringe containing a composition comprising fulvestrant, a pharmaceutically acceptable alcohol(s), a cosolvent(s) comprising benzyl benzoate, polyethylene glycol, and/or propylene glycol, a release controlling lipophilic vehicle comprising castor oil; and, optionally, an antioxidant and/or a surfactant comprising a hydrophilic surfactant and/or a lipophilic surfactant (e.g., poloxamer 188 and SPAN® 20).

In certain embodiments, the disclosure provides a kit comprising at least one sustained release dosage form of an injectable pharmaceutical composition comprising fulvestrant, a pharmaceutically acceptable alcohol(s); a cosolvent(s) comprising benzyl benzoate, polyethylene glycol, and/or propylene glycol; a lipophilic vehicle comprising castor oil; and, optionally, an antioxidant and/or a surfactant comprising a hydrophilic surfactant and/or a lipophilic surfactant; and instructions for therapeutic administration of at least one dosage form. In certain embodiments, the kit comprises prefilled syringes containing fulvestrant in a concentration of, e.g., 500 mg/5 ml, or 1000 mg/5 ml.

In certain embodiments, it surprisingly has been observed that a composition comprising fulvestrant at 100 mg/ml concentration; a pharmaceutically acceptable alcohol(s); a cosolvent(s) comprising propylene glycol, benzyl benzoate, and/or polyethylene glycol; a hydrophilic surfactant; a lipophilic surfactant; a release-controlling lipophilic vehicle comprising castor oil; and, optionally, an antioxidant may provide comparable or improved stability (with respect to fulvestrant degradation, e.g., oxidation) compared to a composition comprising fulvestrant at 50 mg/ml concentration; a pharmaceutically acceptable alcohol(s); benzyl benzoate; and castor oil. The term “stability,” as used herein with respect to a composition, is meant to encompass any characteristics of a composition that may be affected by storage conditions including, without limitation, total impurities, fulvestrant degradation products, specific optical rotation, optical purity, water content, appearance, viscosity, sterility, color, and clarity. Methods of determining stability of a composition with respect to the above parameters include, without limitation, high performance liquid chromatography (HPLC), or thin layer chromatography (TLC).

In certain embodiments of the disclosure, the composition is stable under long-term and accelerated storage conditions. In certain embodiments, the composition is stable under long-term storage conditions for a period of at least 3 months, 6 months, 12 months, 18 months, 2 years, 3 years, or 4 years. In certain embodiments, the composition is stable under accelerated storage conditions for a period of at least 3 months or 6 months. In certain embodiments, the composition of the disclosure, upon storage at 5±3° C. (long-term storage conditions) for a period of at least one year, or upon storage at 25° C. (accelerated storage conditions) for a period of at least three months, contains not more than 2% of total impurities. In certain embodiments, the composition of the disclosure, upon storage at 5±3° C. for a period of at least one year, or upon storage at 25° C. for a period of at least three months, contains not more than about 3.0% of total impurities, e.g., not more than about 2.5%, 2.0%, 1.9%, 1.8%, 1.7%, 1.6%, 1.5%, 1.4%, 1.3%, 1.2%, 1.1%, 1%, 0.5%, 0.2%, or 0.1% of total impurities.

In certain embodiments of the disclosure, oxidative degradation products of fulvestrant include fulvestrant sulfone, 6-keto fulvestrant, and fulvestrant extended. In certain embodiments, the composition of the disclosure upon storage under long-term or accelerated storage conditions contains any one of fulvestrant sulfone, 6-keto fulvestrant, and fulvestrant extended in an amount of not more than about 0.2%, e.g., not more than about 0.18%, 0.16%, 0.14%, 0.12%, 0.1%, 0.08%, 0.06%, or 0.04%.

The composition of the disclosure is suitable for administration to a subject to treat or prevent a disease or condition. In certain embodiments, the disease or condition is benign or malignant disease of the breast or reproductive tract. In certain embodiments, the disease or condition is breast cancer.

In certain embodiments, the volume of the composition comprising 100 mg/ml fulvestrant administered per IM injection is 5 ml or less; e.g., 4.5, 4, 3.5, 3, or 2.5 ml. In certain embodiments, a single injection is administered IM to the subject to deliver about 500 mg of fulvestrant. In certain embodiments, more than one injection is administered IM to the subject, for example, to deliver 500 mg or more of fulvestrant. In certain embodiments, the volume of composition administered per injection is about 5 ml. In certain embodiments, the volume of composition administered as a single IM injection per dosing period (e.g., dosing interval) is about 5 ml.

In certain embodiments, a composition of the disclosure can be administered as monotherapy, or can be administered as a combination therapy comprising administration of fulvestrant and one or more additional drugs. In certain embodiments, if it is a component of a combination therapy, a composition of the disclosure can be administered prior to, substantially concurrent with, or after the administration of one or more additional drugs. In certain embodiments, the fulvestrant dosage form of the disclosure is administered in combination with palbociclib in women with disease progression after endocrine therapy.

In certain embodiments, a composition is administered as a second-line therapy, e.g., following anti-estrogen therapy or following other endocrine therapy. In certain embodiments of the disclosure, the composition can be administered as a first-line therapy.

### Methods

In certain embodiments, the disclosure provides several methods of treatment, manufacture, etc., closely related to the sustained release fulvestrant compositions suitable for IM administration.

In certain embodiments, the disclosure provides a method of improving solubility and stability of fulvestrant in a solution, comprising adding ethanolic solutions of SPAN® 20, poloxamer 188, and, optionally, dl-α-tocopherol, to a vessel containing fulvestrant, alcohol and benzyl alcohol; stirring the resulting mixture to obtain a clear solution; adding cosolvent(s) comprising benzyl benzoate, polyethylene glycol, propylene glycol, and/or a mixture of PEG and PG, and a weighed amount of castor oil to obtain a final volume. In certain embodiments, the resulting composition is filled into a 5 ml syringe. In certain embodiments, the composition upon storage for at least twelve months, under standard storage conditions, contains total impurities in an amount of not more than 2%.

In certain embodiments, the disclosure provides a therapeutic method comprising administering IM to a subject in need thereof, a pharmaceutical composition comprising fulvestrant, a pharmaceutically acceptable alcohol, a cosolvent comprising propylene glycol and/or benzyl benzoate, a lipophilic vehicle comprising castor oil, and, optionally, an antioxidant and/or a surfactant comprising a lipophilic surfactant and/or a hydrophilic surfactant; wherein administration of the composition achieves reduction in tissue damage (measured by, e.g., blood levels of creatine kinase) due to adverse events at the injection site, as compared to that caused by injections of FASLODEX®. In certain embodiments, the adverse events comprise reduction in pain, reduction in discomfort, reduction in inflammation, and/or reduction in hemorrhage.

In certain embodiments, the disclosure provides a dosing regimen comprising IM administration to a subject a pharmaceutical composition; wherein the pharmaceutical composition comprises fulvestrant, a pharmaceutically acceptable alcohol, a cosolvent comprising benzyl benzoate, polyethylene glycol, and/or propylene glycol, a release controlling oil, and, optionally, an antioxidant and/or a mixture of at least one lipophilic surfactant and/or at least one hydrophilic surfactant; and wherein the dosing regimen comprises administering a single injection from a prefilled syringe on Day 1, Day 15, Day 29, and a single injection from a prefilled syringe once monthly thereafter, wherein the single prefilled syringe comprises the pharmaceutical composition in a configuration of 500 mg/5 ml. In certain embodiments, the composition provides a therapeutically significant blood plasma fulvestrant concentration of at least about 2.5 ng/ml for at least about two weeks after injection. In certain embodiments, the subject is a postmenopausal woman with hormonal-dependent benign or malignant disease of the breast or reproductive tract.

In certain embodiments, the disclosure provides methods of treatment of hormonal-dependent benign or malignant diseases of breast and/or reproductive tract in a subject, comprising administering IM to said subject, a pharmaceutical composition comprising fulvestrant; a pharmaceutically acceptable alcohol(s) comprising ethanol and/or benzyl alcohol; a cosolvent(s) comprising propylene glycol, polyethylene glycol, benzyl benzoate, or a mixture thereof, a lipophilic vehicle comprising castor oil, and, optionally, an antioxidant and/or a surfactant comprising a lipophilic surfactant and/or a hydrophilic surfactant.

In certain embodiments, the disclosure provides a method for degrading estrogen receptors in a subject, comprising administering IM to said subject, a pharmaceutical composition comprising fulvestrant; a pharmaceutically acceptable alcohol(s) comprising ethanol and/or benzyl alcohol; a cosolvent(s) comprising propylene glycol, polyethylene glycol, benzyl benzoate, or a mixture thereof; a lipophilic vehicle comprising castor oil, and, optionally, an antioxidant and/or a surfactant comprising a lipophilic surfactant and/or a hydrophilic surfactant.

In certain embodiments, the disclosure provides a therapeutic method comprising IM administration of, to a subject in need thereof, a pharmaceutical composition comprising fulvestrant; a pharmaceutically acceptable alcohol(s) comprising ethanol and/or benzyl alcohol; a cosolvent(s) comprising propylene glycol, polyethylene glycol, benzyl benzoate, or a mixture thereof; a lipophilic vehicle comprising castor oil, and, optionally, an antioxidant and/or a surfactant comprising a lipophilic surfactant and/or a hydrophilic surfactant, wherein administration of the composition achieves reduction in adverse events at the injection site(s) as compared to intramuscular injection of FASLODEX®.

In certain embodiments, the disclosure provides a method for improving patient compliance in treating HR-positive metastatic breast cancer comprising administering a pharmaceutical composition comprising fulvestrant as a single IM injection, wherein the composition comprises fulvestrant, a pharmaceutically acceptable alcohol(s), a cosolvent, a lipophilic surfactant, a hydrophilic surfactant, and a lipophilic vehicle comprising castor oil; wherein the composition has a concentration of 100 mg/ml.

In certain embodiments, the disclosure provides a method of stabilizing a fulvestrant composition. The method includes forming a clear solution comprising fulvestrant, a pharmaceutically acceptable alcohol (e.g., a mixture of benzyl alcohol and ethanol), a hydrophilic surfactant, a lipophilic surfactant, and an antioxidant; and adding a cosolvent (e.g., benzyl benzoate and/or propylene glycol) and a weighed amount of castor oil to the clear solution to obtain a final volume.

In certain embodiments, the disclosure provides a method of manufacturing an injectable pharmaceutical composition. The method comprises mixing in a suitable vessel fulvestrant, a pharmaceutically acceptable alcohol(s) (e.g., a mixture of benzyl alcohol and ethanol), a hydrophilic surfactant, a lipophilic surfactant, and, optionally, an antioxidant to obtain a clear solution; and adding a cosolvent (e.g., benzyl benzoate and/or propylene glycol) and a weighed amount of castor oil to the clear solution to obtain a final volume.

In certain embodiments, the disclosure provides a method of treating a condition comprising a benign or malignant disease of the breast or reproductive tract, for example, breast cancer. In certain embodiments, the method comprises administration to an individual in need of such treatment by IM injection a sustained release pharmaceutical formulation comprising at least 50 mg/ml of fulvestrant (e.g., 100 mg/ml, 150 mg/ml), a pharmaceutically acceptable alcohol, e.g., benzyl alcohol and/or ethanol, a cosolvent comprising benzyl benzoate, polyethylene glycol, and/or propylene glycol, a release-controlling lipophilic vehicle comprising castor oil; a surfactant comprising a hydrophilic surfactant and/or a lipophilic surfactant; and, optionally, an antioxidant.

In certain embodiments, the disclosure provides a method of treating hormone receptor positive metastatic breast cancer in a subject in need thereof. The method comprises administering a therapeutically effective amount of a composition of the disclosure to a subject, thereby treating hormone receptor-positive metastatic breast cancer in the subject. In certain embodiments, the method comprises administering a therapeutically effective amount of a composition of the disclosure to a subject, thereby treating hormone receptor-positive, human epidermal growth factor receptor 2 (HER2)-negative advanced or metastatic breast cancer in the subject. Preferably, the composition is administered to the subject by IM injection. In certain embodiments, the therapeutically effective amount of the composition can be administered in a single injection, or divided into two or more injections. In certain embodiments, the subject comprises postmenopausal women with disease progression following antiestrogen therapy, e.g., aromatase inhibitor therapy. In certain embodiments, the subject comprises women with disease progression following endocrine therapy.

In certain embodiments, the disclosure provides a method for identifying a sub-population of patients for treatment, the sub-population comprising women with hormone receptor-positive, human epidermal growth factor receptor 2 (HER2)-negative, advanced or metastatic breast cancer, with disease progression after a first endocrine therapy, the treatment comprising administering a composition of the disclosure. In certain embodiments, the composition of the disclosure is administered in combination with palbociclib.

In certain embodiments, the disclosure provides a method of degradation of estrogen receptors to block cellular signaling for estrogen synthesis, thereby treating hormone receptor-positive, human epidermal growth factor receptor 2 (HER2)-negative advanced or metastatic breast cancer in the subject, the method comprising administering a therapeutically effective amount of a composition of the disclosure to the subject.

In certain embodiments, the disclosure provides a method of degradation of estrogen receptors to block cellular signaling for estrogen synthesis, thereby treating hormone receptor (HR)-positive metastatic breast cancer in postmenopausal women with disease progression following antiestrogen therapy as monotherapy, the method comprising administering a therapeutically effective amount of a composition of the disclosure to the subject.

In certain embodiments, the disclosure provides a method for decreasing the frequency by which a muscle site is subject to intramuscular injection. The method comprises administering by IM injection an improved fulvestrant pharmaceutical composition comprising fulvestrant, a pharmaceutically acceptable alcohol(s), a cosolvent, a lipophilic vehicle comprising castor oil, and, optionally, an antioxidant and/or a surfactant comprising a lipophilic surfactant and/or a hydrophilic surfactant, wherein the concentration of fulvestrant in the composition is at least 100 mg/ml.

The following Examples illustrate the disclosure in a nonlimiting manner. Unless indicated to the contrary, the numerical parameters set forth herein may vary depending upon the desired properties sought to be obtained by the present disclosure.

### EXAMPLES

### Example 1: Method of Manufacture of Fulvestrant Formulation Compositions

Prototype formulation compositions A-Q of the disclosure are described in Tables 4 and 5.

TABLE 4

| | Prototype Formulation Compositions (% w/w) | | | | | | | |
|---|---|---|---|---|---|---|---|---|
| Ingredients | A | B | C | D | E | F | G | H |
| Fulvestrant | 10.0 | 10.0 | 10.0 | 10.0 | 10.0 | 10.0 | 10.0 | 10.0 |
| Benzyl benzoate | — | 5.0 | — | 2.0 | 1.5 | — | — | 5.0 |
| Propylene glycol (PG) | — | — | 5.0 | — | 1.5 | 2.0 | 2.5 | 2.5 |
| Polyethylene glycol | — | — | — | — | — | — | 2.5 | 2.5 |
| (PEG) 400 | | | | | | | | |
| Benzyl alcohol | 10.0 | 10.0 | 10.0 | 10.0 | 10.0 | 10.0 | 10.0 | 10.0 |
| Ethanol | 18.0 | 18.0 | 18.0 | 18.0 | 18.0 | 18.0 | 18.0 | 18.0 |
| Sorbitan monolaurate | 0.20 | 0.20 | 0.20 | 0.20 | 0.20 | 0.20 | — | — |
| Poloxamer 188 | 0.20 | 0.20 | 0.20 | 0.20 | 0.20 | 0.20 | 0.20 | 0.20 |
| dl-α- Tocopherol | 0.06 | 0.06 | 0.06 | 0.06 | 0.06 | 0.06 | 0.06 | 0.06 |
| Castor oil | Qs to | Qs to | Qs to | Qs to | Qs to | Qs to | Qs to | Qs to |
| | 100 g | 100 g | 100 g | 100 g | 100 g | 100 g | 100 g | 100 g |

TABLE 5

| | Prototype Formulation Compositions (% w/w) | | | | | | | | |
|---|---|---|---|---|---|---|---|---|---|
| Ingredients | I | J | K | L | M | N | O | P | Q |
| Fulvestrant | 10.0 | 10.0 | 10.0 | 10.0 | 10.0 | 10.0 | 10.0 | 10.0 | 10.0 |
| Benzyl benzoate | 5.0 | 3.0 | 5.0 | 5.0 | 7.5 | 3.0 | 0.9 | 5.0 | 15.0 |
| PG | 2.5 | — | — | — | — | — | — | — | — |
| PEG 400 | 2.5 | — | — | — | — | 2.0 | 4.1 | 2.0 | — |
| Benzyl alcohol | 10.0 | 10.0 | 10.0 | 10.0 | 10.0 | 10.0 | 10.0 | 10.0 | 10.0 |
| Ethanol | 18.0 | 18.0 | 15.0 | 15.0 | 15.0 | 18.0 | 15.0 | 15.0 | 10.0 |
| Sorbitan monolaurate | 0.20 | 0.25 | 0.25 | 0.20 | 0.25 | 0.25 | 0.25 | 0.25 | — |
| Poloxamer 188 | — | 0.10 | 010 | 0.20 | 0.10 | 0.10 | 0.10 | 0.10 | — |
| dl-α- Tocopherol | 0.06 | 0.06 | 0.06 | 0.06 | 0.06 | 0.06 | 0.06 | 0.06 | — |
| Castor oil | Qs to | Qs to | Qs to | Qs to | Qs to | Qs to | Qs to | Qs to | Qs to |
| | 100 g | 100 g | 100 g | 100 g | 100 g | 100 g | 100 g | 100 g | 100 g |

Formulation compositions A-Q were prepared according to the following general procedure. As per formulation composition A-Q, ethanolic solutions of SPAN® 20, dl-α-tocopherol, and poloxamer 188) were added to a vessel containing fulvestrant, alcohol and benzyl alcohol. The resulting mixture was stirred to obtain a clear solution. To the resulting clear solution, a cosolvents) comprising benzyl benzoate, polyethylene glycol, and/or propylene glycol, and a weighed amount of castor oil was added to obtain a final weight. The vessel was purged with nitrogen and the solution was filtered through a filter with a pore diameter of 0.22 μm. The resulting filtered solution was filled into 5 ml syringes.

### Example 2: Method of Manufacture of Fulvestrant Formulation Compositions

Prototype formulation compositions R-Y of the disclosure are described in Table 6.

TABLE 6

| | Prototype Formulation Compositions (% w/w) | | | | | | | |
|---|---|---|---|---|---|---|---|---|
| Ingredients | R | S | T | U | V | W | X | Y |
| Fulvestrant | 10.0 | 10.0 | 13.0 | 13.0 | 10.0 | 10.0 | 10.0 | 10.0 |
| Benzyl benzoate | — | — | — | — | — | — | — | — |
| PG | — | 2.5 | — | 5.0 | 3.75 | 2.5 | — | 5.0 |
| PEG 400 | — | 2.5 | 5.0 | — | 3.75 | 2.5 | 5.0 | — |
| Benzyl alcohol | 10.0 | 10.0 | 10.0 | 10.0 | 7.5 | 10.0 | 10.0 | 10.0 |
| Ethanol | 18.0 | 15.0 | 15.0 | 15.0 | 15.0 | 15.0 | 15.0 | 15.0 |
| Sorbitan monolaurate | 0.20 | — | — | — | — | — | — | — |
| Poloxamer 188 | 0.20 | 0.10 | 0.10 | 0.10 | 0.20 | 0.50 | 0.50 | 0.50 |
| dl-α- Tocopherol | 0.06 | 0.06 | 0.06 | 0.06 | 0.06 | 0.06 | 0.06 | 0.06 |
| Castor oil | Qs to | Qs to | Qs to | Qs to | Qs to | Qs to | Qs to | Qs to |
| | 100 g | 100 g | 100 g | 100 g | 100 g | 100 g | 100 g | 100 g |

Formulation compositions R-Y were prepared according to procedures similar to the general procedures described in Example 1.

### Example 3: Method of Manufacture of Fulvestrant Formulation Compositions

Prototype formulation compositions Z1-Z39 of the disclosure are described in Tables 7-12.

TABLE 7

| | Prototype Formulation Compositions (% w/w) | | | | |
|---|---|---|---|---|---|
| Ingredients | Z1 | Z2 | Z3 | Z4 | Z5 |
| Fulvestrant | 10.0 | 10.0 | 10.0 | 10.0 | 10.0 |
| Benzyl benzoate | — | — | 5.00 | — | — |
| PG | 2.5 | 2.15 | — | 5.0 | 2.15 |
| PEG 400 | 2.5 | — | — | — | — |
| Benzyl alcohol | 10.0 | 10.0 | 7.5 | 10.0 | 10.0 |
| Ethanol | 18.0 | 17.7 | 18.0 | 18.0 | 17.7 |
| Sorbitan monolaurate | 0.20 | 0.20 | — | — | 0.20 |
| Poloxamer 188 | — | 0.20 | 0.20 | — | 0.20 |
| dl-α-Tocopherol | 0.06 | — | 0.06 | 0.06 | 0.24 |
| Castor oil | Qs to | Qs to | Qs to | Qs to | Qs to |
| | 100 g | 100 g | 100 g | 100 g | 100 g |

TABLE 8

| | Prototype Formulation Compositions (% w/w) | | | | | |
|---|---|---|---|---|---|---|
| Ingredients | Z6 | Z7 | Z8 | Z9 | Z10 | Z11 |
| Fulvestrant | 10.0 | 10.0 | 10.0 | 10.0 | 10.0 | 10.0 |
| Benzyl benzoate | — | — | 7.0 | 5.0 | — | — |
| PG | 5.0 | 5.0 | — | — | 5.0 | 2.1 |
| PEG 400 | — | — | — | — | — | — |
| Benzyl alcohol | 10.0 | 10.0 | — | 10.0 | 10.0 | 10.0 |
| Ethanol | 18.0 | 18.0 | 26.0 | 18.0 | 18.0 | 17.3 |
| Sorbitan monolaurate | 0.20 | 0.20 | 0.25 | — | 0.20 | 0.20 |
| Poloxamer 188 | 0.20 | 0.20 | 0.10 | — | — | 0.20 |
| dl-α- Tocopherol | — | — | 0.06 | 0.06 | 0.06 | 0.06 |
| Castor oil | Qs to | Qs to | Qs to | Qs to | Qs to | Qs to |
| | 100 g | 100 g | 100 g | 100 g | 100 g | 100 g |

TABLE 9

| | Prototype Formulation Compositions (% w/w) | | | | | | | |
|---|---|---|---|---|---|---|---|---|
| Ingredients | Z12 | Z13 | Z14 | Z15 | Z16 | Z17 | Z18 | Z19 |
| Fulvestrant | 10.0 | 10.0 | 10.0 | 10.0 | 10.0 | 10.0 | 10.0 | 10.0 |
| Benzyl benzoate | 0.5 | — | — | 2.0 | 2.0 | 3.0 | — | — |
| PG | 2.3 | 2.15 | 3.0 | 2.0 | 3.0 | 2.0 | 2.3 | 2.6 |
| PEG 400 | — | — | — | — | — | — | — | — |
| Benzyl alcohol | 10.0 | 10.0 | 10.0 | 10.0 | 10.0 | 10.0 | 10.0 | 10.0 |
| Ethanol | 17.5 | 17.7 | 18.0 | 18.0 | 18.0 | 18.0 | 17.5 | 17.5 |
| Sorbitan monolaurate | 0.20 | 0.20 | 0.20 | 0.20 | 0.20 | 0.20 | 0.20 | 0.20 |
| Poloxamer 188 | 0.20 | 0.20 | 0.20 | 0.20 | 0.20 | 0.20 | 0.20 | 0.20 |
| dl-α- Tocopherol | 0.06 | 0.12 | 0.06 | 0.06 | 0.06 | 0.06 | 0.06 | 0.06 |
| Castor oil | Qs to | Qs to | Qs to | Qs to | Qs to | Qs to | Qs to | Qs to |
| | 100 g | 100 g | 100 g | 100 g | 100 g | 100 g | 100 g | 100 g |

TABLE 10

| | Prototype Formulation Compositions (% w/w) | | | | | | | |
|---|---|---|---|---|---|---|---|---|
| Ingredients | Z20 | Z21 | Z22 | Z23 | Z24 | Z25 | Z26 | Z27 |
| Fulvestrant | 10.0 | 10.0 | 10.0 | 10.0 | 10.0 | 10.0 | 10.0 | 10.0 |
| Benzyl benzoate | 5.0 | — | — | — | — | — | 10.0 | 10.0 |
| PG | 2.5 | — | — | — | — | 5.0 | — | — |
| PEG 400 | 2.5 | — | — | — | — | — | — | — |
| Benzyl alcohol | 10.0 | 10.0 | 10.0 | 10.0 | 10.0 | 10.0 | 15.0 | 15.0 |
| Ethanol | 20.0 | 15.0 | 15.0 | 15.0 | 17.0 | 17.0 | 10.0 | 10.0 |
| Sorbitan monolaurate | — | — | — | — | — | — | — | — |
| Poloxamer 188 | 0.20 | — | 0.30 | 0.30 | 0.20 | — | — | — |
| Polyvinyl pyrrolidone | — | 1.0 | — | 0.7 | — | 0.5 | 10.0 | 5.0 |
| K12 | | | | | | | | |
| dl-α- Tocopherol | 0.06 | 0.06 | 0.06 | 0.06 | 0.06 | 0.06 | — | — |
| Castor oil | Qs to | Qs to | Qs to | Qs to | Qs to | Qs to | Qs to | Qs to |
| | 100 g | 100 g | 100 g | 100 g | 100 g | 100 g | 100 g | 100 g |

TABLE 11

| | Prototype Formulation Compositions (% w/w) | | | | | | | | |
|---|---|---|---|---|---|---|---|---|---|
| Ingredients | Z28 | Z29 | Z30 | Z31 | Z32 | Z33 | Z34 | Z35 | Z36 |
| Fulvestrant | 10.0 | 10.0 | 10.0 | 10.0 | 10.0 | 10.0 | 10.0 | 10.0 | 10.0 |
| Benzyl benzoate | 15.0 | 15.0 | — | 15.0 | — | 15.0 | — | 20.0 | 10.0 |
| PG | — | — | 2.8 | — | 4.0 | — | 2.8 | — | — |
| PEG 400 | — | — | — | — | — | — | | 10.0 | 10.0 |
| Benzyl alcohol | 10.0 | 10.0 | 10.0 | 10.0 | 10.0 | — | 10.0 | 15.0 | — |
| Ethanol | 10.0 | 10.0 | 18.0 | 10.0 | 18.0 | 20.0 | 20.0 | — | 20.0 |
| Sorbitan monolaurate | — | — | 0.2 | — | 0.2 | — | 0.2 | — | — |
| Poloxamer 188 | — | 5.0 | 0.2 | 10.0 | 0.2 | — | 0.2 | — | — |
| Polyvinyl pyrrolidone | 2.0 | — | — | — | — | 0.5 | — | — | — |
| K12 | | | | | | | | | |
| dl-α- Tocopherol | — | — | — | — | — | — | — | — | — |
| Castor oil | Qs to | Qs to | Qs to | Qs to | Qs to | Qs to | Qs to | Qs to | Qs to |
| | 100 g | 100 g | 100 g | 100 g | 100 g | 100 g | 100 g | 100 g | 100 g |

TABLE 12

| Ingredients | Z37 | Z38 | Z39 | Z40 |
|---|---|---|---|---|
| Fulvestrant | 10.0 | 10.0 | 10.0 | 10.0 |
| Benzyl benzoate | 15.0 | 15.0 | 15.8 | — |
| PG | — | — | — | 4.0 |
| PEG 400 | — | — | — | — |
| Benzyl alcohol | 10.0 | 10.0 | 20.0 | 10.0 |
| Ethanol | 10.0 | 10.0 | — | 20.0 |
| Sorbitan monolaurate | — | — | — | 0.2 |
| Poloxamer 188 | — | 7.0 | — | 0.2 |
| Polyvinyl pyrrolidone | 5.0 | — | — | — |
| K12 | | | | |
| dl-α-Tocopherol | — | — | — | — |
| Castor oil | Qs to | Qs to | Qs to | Qs to |
| | 100 g | 100 g | 100 g | 100 g |

Formulation compositions Z1-Z40 were prepared according to procedures similar to the general procedures described in Example 1.

### Example 4: Determination of Solubility of Prototype Formulation Compositions

Solubility in the formulation vehicle was determined to ensure that the product can achieve and maintain the desired solubility of fulvestrant for the intended duration. Excess drug was added to the formulation vehicle containing the desired amount of solvent, cosolvent, surfactants, and castor oil, and the samples were allowed to stir for 48 hours at ambient conditions. The dissolved amount of fulvestrant was measured by HPLC after centrifugation and filtration of the sample.

Solubility of fulvestrant in various dosage forms of the disclosure, measured at room temperature, was in the range of about 130-180 mg/ml.

### Example 5: Determination of Viscosity of Prototype Formulation Compositions

Based on the significant differences in the viscosities of cosolvents and lipid vehicle (Table 3), it is expected that their relative concentrations in the formulations can be critical not only for solubility but also for stability and performance, e.g., release profile of the formulations.

Viscosities of various dosage forms of the disclosure containing fulvestrant at 100 mg/ml, and FASLODEX® containing fulvestrant at 50 mg/ml, were determined using a Brookfield viscometer. The viscosities of the dosage forms of the disclosure were in the range of about 80 to about 120 cp, and the viscosity of FASLODEX® was 96.6 cp.

### Example 6: Determination of Storage Stability of Prototype Formulation Compositions

TABLE 13

| | Formulation 1 | Formulation 2 | Formulation 3 |
|---|---|---|---|
| Ingredients | (% w/v) | (% w/v) | (% w/v) |
| Fulvestrant | 10.0 | 10.0 | 10.0 |
| Propylene glycol | 2.15 | 2.15 | 2.15 |
| Benzyl alcohol | 10.0 | 10.0 | 10.0 |
| Ethanol | 17.7 | 17.7 | 17.7 |
| Sorbitan | 0.2 | 0.2 | 0.2 |
| monolaurate | | | |
| Poloxamer 188 | 0.2 | 0.2 | 0.2 |
| dl-α-Tocopherol | 0.24 | 0.12 | 0.0 |
| Castor oil | Qs to 1 ml | Qs to 1 ml | Qs to 1 ml |

TABLE 14

| | Assay % dl-α-Tocopherol | | |
|---|---|---|---|
| Storage conditions | Formulation 1 | Formulation 2 | Formulation 3 |
| Initial | 98.7 | 96.0 | ND |
| 5° C. | 89.4 | 73.1 | ND |
| Room temp | 54.2 | 19.1 | ND |
| 40° C./75% RH | 39.5 | 6.3 | ND |

TABLE 15

| | | Storage | Total |
|---|---|---|---|
| | Storage condition | time | impurities |
| | | (weeks) | (% w/w) |
| Formulation 1 | 5 °C. | 4 | 0.525 |
| | RT | 4 | 0.614 |
| | 40° C./75% RH | 4 | 0.748 |
| Formulation 2 | 5° C. | 3 | 0.502 |
| | RT | 3 | 0.502 |
| | 40° C./75% RH | 3 | 0.723 |
| | | 4 | 0.893 |
| Formulation 3 | 5° C. | 4 | 0.520 |
| | RT | 4 | 0.529 |
| | 40° C./75% RH | 4 | 0.780 |

Three-week and four-week storage stabilities, as per Table 15, of Formulations 1-3 were determined under standard storage conditions of 5° C., storage at room temperature, and storage at 40° C./75% RH. The total impurities were less than 1% at the end of three- or four-week storage at 5° C., RT, and 40° C./75% RH.

It was observed that the presence of antioxidant (at the two levels of 0.24% and 0.12%) or a complete absence of the antioxidant did not result in significant differences in storage stability of the compositions.

Table 14 shows assay % of dl-α-Tocopherol for Formulations 1-3 under various storage conditions. It was surprisingly observed that dl-α-Tocopherol, which was used as a stabilizer, was itself unstable and susceptible to degradation under various storage conditions.

### Example 7: Determination of Stability of Prototype Formulation Compositions

Preliminary formulation stability was performed in vials to provide an initial assessment. For preliminary stability assessment, the formulations were filled into vials after appropriately flushing the headspace with nitrogen, and vials were stoppered and crimped. The packaged product was stored at 5° C. (intended storage conditions), and at 25° C./60% RH (accelerated (stress) conditions) for durations of two and four weeks. The drug content and the major degradation products, 6-keto fulvestrant and fulvestrant 9 sulfonate, were determined using HPLC.

The tested formulation compositions maintained their physical and chemical stability for two weeks. No major concerns were observed with regard to the stability of the product, i.e., the total impurities were below 1%, and each of the individual impurities was below 0.2%. 6-keto fulvestrant levels were comparable to FASLODEX®. The level of total impurities in the tested formulations of the disclosure at intended long-term storage conditions was between 0.5% and 1%, and at accelerated conditions was also between 0.5% and 1%. The level of 6-keto fulvestrant at intended long-term storage conditions was between 0.05% and 0.1%, and at accelerated conditions was also between 0.05% and 0.1%. The amount of fulvestrant sulfone impurity at intended storage conditions and at accelerated conditions at two weeks was below 0.2%.

### Example 8: Pharmacokinetic Studies in Dogs

Pharmacokinetic studies were conducted to compare the exposure of several dosage forms of the disclosure to that of FASLODEX® at a dose of 25 mg/kg in male beagle dogs by intramuscular injection. Considering a 60 kg average weight for the adult human, this dose represents approximately a 2.5-fold higher dose as compared to the projected dose in human. FASLODEX® was administered at a dose of 250 mg/5 ml, whereas tested dosage forms of the disclosure were injected at a dose of 250 mg/2.5 ml. Blood samples were collected pre-dose and post-dose at different time intervals for up to 28 days. Observations at the injection site indicated some that there was detectable swelling at the injection site at approximately two hours post-dose in the test and RLD groups, and the swelling subsided over the next few hours. Some injection sites exhibited swelling at later time points in the case of the test formulations, with no other major observations.

Based on the pharmacokinetic profiles, the fulvestrant formulations of the disclosure provided exposures in broad ranges similar to those for FASLODEX®. In dog studies (presenting the ranges of mean values for several formulations), the tested formulations of the disclosure achieved for fulvestrant a Cₘₐₓ (ng/ml) range of 19.2±5.41 to 90.5±95.5, a dose normalized AUC_{0-28 days} (hr kg ng/ml/mg) range of 288±144 to 580±136, and a T_{1/2} (hr) range of 197±34.9 to 391±186.

### Example 9: IM Tolerance Studies in Rabbits

Injection site reactions and histopathology of formulation compositions of the disclosure were compared to that of FASLODEX®. FASLODEX® was administered as two IM injections at two injection sites, a total dose of 50 mg given as 25 mg/site (0.5 ml at each of two injection sites), whereas the test formulations of the disclosure were injected as a single IM injection at a dose of 50 mg (0.5 ml at one injection site). Blood samples were collected post-dose at time intervals for up to 50 days.

Observations were made at 10, 30, and 50 days after intramuscular administration of FASLODEX® and two fulvestrant formulations of the disclosure in New Zealand white rabbits. The following observations were made.
No treatment-related mortality and morbidity detected in rabbits with the use of test formulations or FASLODEX®
No treatment-related significant changes in body weight detected in rabbits with the use of test formulations or FASLODEX®
Increase in creatine kinase levels detected in treated rabbits at ˜6 and ˜24 hours post-dose. The creatine kinase level decreased to values within normal range in all treated rabbits within 48 hours.

1-58. (canceled)

59. A sustained release pharmaceutical composition suitable for intramuscular administration comprising fulvestrant, a pharmaceutically acceptable alcohol, propylene glycol, a surfactant, and a lipophilic vehicle comprising castor oil, wherein the composition is substantially free of antioxidant and benzyl benzoate.

60. The composition of claim 59, wherein the composition comprises fulvestrant at a concentration of about 5% to about 20% w/v of the composition.

61. The composition of claim 59, wherein the composition comprises fulvestrant at a concentration of at least about 100 mg/ml in a single 5 ml injection.

62. The composition of claim 61, wherein the composition provides a fulvestrant concentration that is therapeutically equivalent to a commercially available fulvestrant product administered at a same total dose delivered in two 5 ml injections.

63. The composition of claim 59, wherein the pharmaceutically acceptable alcohol comprises ethanol, benzyl alcohol, or a mixture thereof.

64. The composition of claim 59, wherein the pharmaceutically acceptable alcohol comprises 5% w/v to about 25% w/v of ethanol.

65. The composition of claim 59, wherein the pharmaceutically acceptable alcohol comprises 5% w/v to about 25% w/v of benzyl alcohol.

66. The composition of claim 63 comprising at least about 5% w/v of benzyl alcohol and at least about 10% w/v of ethanol.

67. The composition of claim 63 comprising at least about 5% w/v of benzyl alcohol and at least about 20% w/v of ethanol.

68. The composition of claim 59, wherein the surfactant comprises a lipophilic surfactant, a hydrophilic surfactant, or a mixture thereof.

69. The composition of claim 59, wherein the surfactant comprises a mixture of a lipophilic surfactant and a hydrophilic surfactant.

70. The composition of claim 68, wherein the lipophilic surfactant is selected from the group consisting of sorbitan trioleate, sorbitan tristearate, sorbitan monooleate, sorbitan monostearate, sorbitan monopalmitate, and sorbitan monolaurate.

71. The composition of claim 70, wherein the lipophilic surfactant is sorbitan monolaurate.

72. The composition of claim 68, wherein the hydrophilic surfactant is selected from the group consisting of polyoxyethylene (20) sorbitan monolaurate, polyoxyethylene (20) sorbitan monopalmitate, polyoxyethylene (20) sorbitan monostearate, polyoxyethylene (20) sorbitan tristearate, polyoxyethylene (20) sorbitan monooleate, and a triblock copolymer of polyoxypropylene and polyoxyethylene (poloxamer 188).

73. The composition of claim 72, wherein the hydrophilic surfactant is a triblock copolymer of polyoxypropylene and polyoxyethylene.

74. The composition of claim 69, wherein the surfactant comprises a mixture of sorbitan monolaurate and a triblock copolymer of polyoxypropylene and polyoxyethylene.

75. The composition of claim 59 comprising at least about 2% w/v of propylene glycol.

76. The composition of claim 59, wherein the composition is physically and chemically stable, with no appreciable precipitation or degradation of fulvestrant, at 5±3° C. for a period of at least six months.

77. The composition of claim 59, wherein following storage of the composition for three months, under long-term or accelerated storage conditions, the total amount of impurities present in the composition is not more than about 2%.

78. The composition of claim 77, wherein long-term storage conditions comprise a temperature of 5±3° C.

79. The composition of claim 77, wherein accelerated storage conditions comprise a temperature of 25±2° C. and a relative humidity of 60±5%.

80. The composition of claim 59, wherein the composition is packaged in a container, and wherein the container comprises a prefilled sterilized clear glass syringe comprising a sterilized plunger stopper, a plunger rod, and a sterilized needle.

81. The composition of claim 80, wherein the syringe comprises fulvestrant at a concentration of at least about 500 mg/5 ml.

82. A sustained release pharmaceutical composition suitable for intramuscular administration comprising fulvestrant, a pharmaceutically acceptable alcohol, propylene glycol, a surfactant, and a lipophilic vehicle comprising castor oil, wherein the composition comprises fulvestrant at a concentration of at least about 100 mg/ml in a single 5 ml injection.

83. The composition of claim 82, wherein the composition is free of benzyl benzoate.

84. The composition of claim 82, wherein the composition is free of antioxidant.

85. The composition of claim 82, wherein the pharmaceutically acceptable alcohol comprises ethanol, benzyl alcohol, or a mixture thereof.

86. The composition of claim 85 comprising at least about 5% w/v of benzyl alcohol and at least about 10% w/v of ethanol.

87. The composition of claim 85 comprising at least about 5% w/v of benzyl alcohol and at least about 20% w/v of ethanol.

88. The composition of claim 82, wherein the surfactant is a mixture of sorbitan monolaurate and a triblock copolymer of polyoxypropylene and polyoxyethylene.

89. The composition of claim 82 comprising at least about 2% w/v of propylene glycol.

90. A sustained release pharmaceutical composition suitable for intramuscular administration comprising: fulvestrant, a pharmaceutically acceptable alcohol, a cosolvent, a lipophilic surfactant, a hydrophilic surfactant, and a lipophilic vehicle comprising castor oil, wherein the composition is free of benzyl benzoate and antioxidant, and wherein the composition provides a fulvestrant dose in a single 5 ml injection that is therapeutically equivalent to a commercially available fulvestrant product at same total dose delivered in two 5 ml injections.

91. The composition of claim 90, wherein the pharmaceutically acceptable alcohol comprises ethanol and benzyl alcohol.

92. The composition of claim 90, wherein the cosolvent is propylene glycol.

93. The composition of claim 90, wherein the hydrophilic surfactant is a triblock copolymer of polyoxypropylene and polyoxyethylene, and the lipophilic surfactant is sorbitan monolaurate.

94. A sustained release pharmaceutical composition suitable for intramuscular administration comprising: fulvestrant, ethanol, benzyl alcohol, propylene glycol, a lipophilic surfactant, a hydrophilic surfactant, and a lipophilic vehicle comprising castor oil, wherein the composition is free of benzyl benzoate and polysorbate 80.

95. A therapeutic method comprising intramuscularly administering to a subject in need thereof a pharmaceutical composition as a single 5 ml injection comprising fulvestrant at a concentration of at least about 100 mg/ml, a pharmaceutically acceptable alcohol, propylene glycol, a surfactant, and a lipophilic vehicle comprising castor oil, wherein the composition provides a fulvestrant concentration that is therapeutically equivalent to a commercially available fulvestrant product at a same total dose delivered in two 5 ml injections, and wherein the administration of the composition achieves a reduction in adverse events, due to reduced number and volume of injections, as compared to intramuscular injections of the commercially available fulvestrant product.

96. The method of claim 95, wherein the composition is free of benzyl benzoate and antioxidant.

97. The method of claim 95, wherein the reduction in adverse events comprises reduction in pain, reduction in inflammation, and/or reduction in hemorrhage.
